(19) United States
(12) Patent Application Publication
(10) Pub. No.: US 2022/0015627 A1
(43) Pub. Date: Jan. 20, 2022

(54) Method and system for detecting blink with proximity sensor

(71) Applicant: Hao-yun Chen, Taichung City (TW)

(72) Inventor: Hao-yun Chen, Taichung City (TW)

(21) Appl. No.: 17/363,025

(22) Filed: Jun. 30, 2021

(30) Foreign Application Priority Data
Jul. 20, 2020 (TW) 109124369

(51) Int. Cl. A61B 3/113; G06F 3/01; G02B 27/00; H05B 47/11; G02B 27/01; H04W 4/80

(52) U.S. Cl. CPC A61B 3/113; G06F 3/013; G02B 27/0093; G02B 2027/0178; G02B 27/017; H04W 4/80; H05B 47/11

(57) ABSTRACT

Disclosed relates to a method and system of determining and recording eye motions. It is a distance measuring photoelectric proximity sensor fixed to the head, which senses the change in distance between the sensor and the eye when the eyelid is opening and closing to detect the eye open and close motion; with its small size and power saving features, it can be used as a head-mounted device for real-time management and long-term recording of eye usage, and is applied to human-machine interfaces to transmit signals generated by user's eye motions to control external components.

### CROSS-REFERENCE TO RELATED APPLICATIONS

The present application claims the benefit of priority of Taiwan Patent Application No. 1019124369, title “Method and system for detecting blink with photoelectric sensor” filed on Jul. 20, 2020.

### BACKGROUND

The major technology of eye motion detection and classification is eye image identification by camera, such as patent TW-201943577 (safe driving monitoring), TW-201915669 (eye detection and identification). Eye motion detection and classification can be better as a human-machine interface in the head-mounted-device (HIVID) field of contention. Such as patent U.S. Pat. Nos. 9,207,760, 9,201,512, TW-201640277, emit from light source, and detect reflected light intensity changes of eye by the light sensor to measure the direction of the eye sight and the blink of the eye motion.

Overuse of eyes induce eye lesions (Ref. US627046 for computer vision syndrome). Since the popularity of mobile phones and PCs, it's even more serious. Students in Junior high school third grade of Taiwan got 89.3% of myopia ratio (press release 1070504 Health Department of Taiwan—myopia is a disease!). Obviously, there is no universal and effective eye monitoring technology to control eye physiological problems.

So far, the eye motion detection and classification technology got low portability because following problems: required lots of detection elements, large volume and high-power consumption caused by image calculation. And the detection data of eye image mixed with eyeballs, eye movements, eyelids, object color, roughness and light source factors, and needed complex interpretation procedures to eliminate noises.

### BRIEF SUMMARY

Disclosed provide a portable detection and classification system of eye's opening and closing states by eyelid movement detection. The system measures the distance change between the sensor and the eye when eyelid opening and closing by a photoelectric proximity sensor (see Proximity sensor, Photoelectric Sensor principle). The proximity sensor with small size and few components is sensitive in short-range distance change, low power consumption and minor noise because it's number of reflected infrared light pulses measuring method. It is miniaturized device, power-efficient and more suitable as the form of wearable device to detect a variety of eye events any time or as a human-machine interface.

Except detect the intentional blink by eye closed time duration as early technology, the disclosure can detect particular eye states such as fully closed or slightly closed of the blink just by determined distance change differential of the eyelid position.

With the calculation of time duration of the particular eye opening and closing state, the disclosure can distinguish eye events such as unconscious blinking, dozing off, resting, opening eyes for a long time, etc., further through the portability feature to track and control the user's physiological state for a long time. It can compute and generate more complex human interface control signals according to various eye events, various particular eye state or combination sequence.

Through the disclosure, it can be combined with other sensor data to provide more physiological detection of the eye. According to research (The myopia boom,

Nature), the duration time of outdoor activities is significantly related to myopia. In one of embodiments of the disclosure, the receiver of the photoelectric proximity sensor can be directly program to add the Ambient Light Sensor data to coordinate with the detection of eye events and track the user's eye environment, no additional sensor is required.

Seasons, days, temperature, air pressure, and humidity changes will affect the evaporation of tears, and then cause dry eye symptoms. In one of embodiments of the disclosure, a long scale timing program is added to eye events detection to perform long-term data analysis to track and understand the physiological condition of the eyes.

### BRIEF DESCRIPTION OF THE DRAWINGS

FIG. 1 illustrate the reflected light pulses between the proximity sensor and the eye states.

FIG. 2 illustrate the example of device mounting mechanism.

FIG. 3 illustrate the example of computer measurement program flow chart.

FIG. 4 illustrate the example of time chart of the number of reflected infrared light pulses with various eye states.

FIG. 5 illustrate the example of device components.

### DETAILED DESCRIPTION

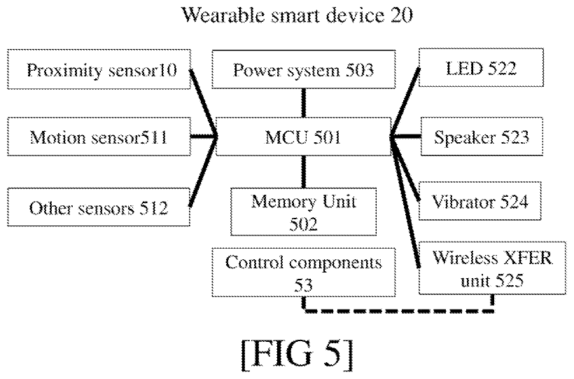

The one of embodiments hardware design refers to the wearable smart device 20 shown in [FIG. 5], which is connected by circuits and mainly includes an MCU microcontroller unit 501, a wireless transmission unit 525, a power supply and battery system 503, and an infrared proximity sensor 10.

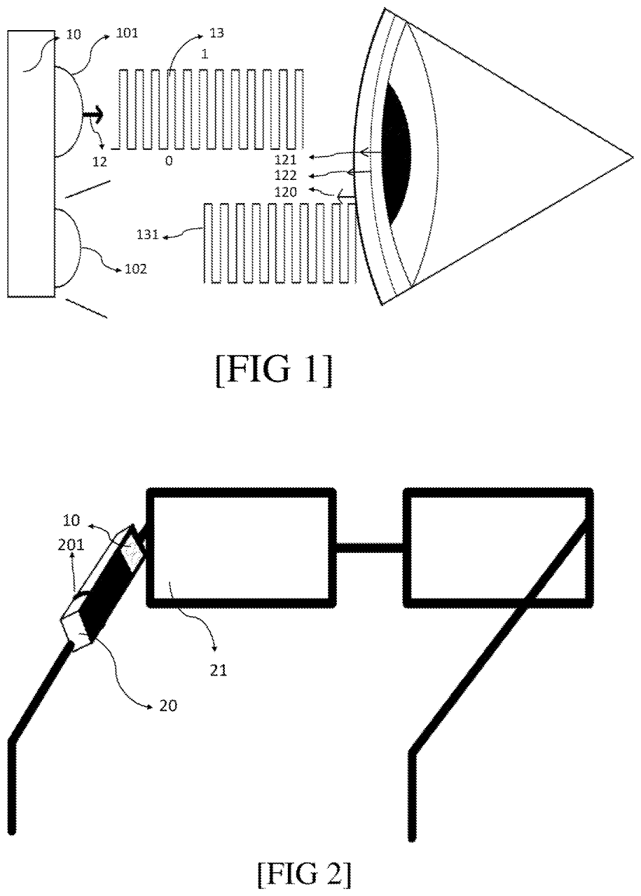

The program instructions stored in the computer-readable medium 502 are processed by the MCU 501 to perform the technical characteristic of the disclosure: as shown in [FIG. 1],the infrared light LED 101 of the photoelectric proximity sensor 10 emits a fixed time duration and number of infrared light pulses 13, the infrared light receiving tube 102 receives and calculates the number of pulses reflected 131 by the eye surface 120˜122 within a fixed time (refer the Proximity sensor, Photoelectric Sensor technical manual for details). The closer the distance with the greater the number of reflected pulses.

In order to prevent the proximity sensor from being interfered, another characteristic of the disclosure is that the proximity sensor has a stable distance related with the eye, which can be fixed in user's head. For a preferred embodiment, refer to [FIG. 2] to fix the device 20 with the sensor 10 to the glasses 21 by at least one elastic band 201, so that the device does not exceed its maximum measuring distance and is not easy to get shift, only the opening and closing of eyelid 120˜121 (0.5-2 mm) will change the distance between eye and the sensor.

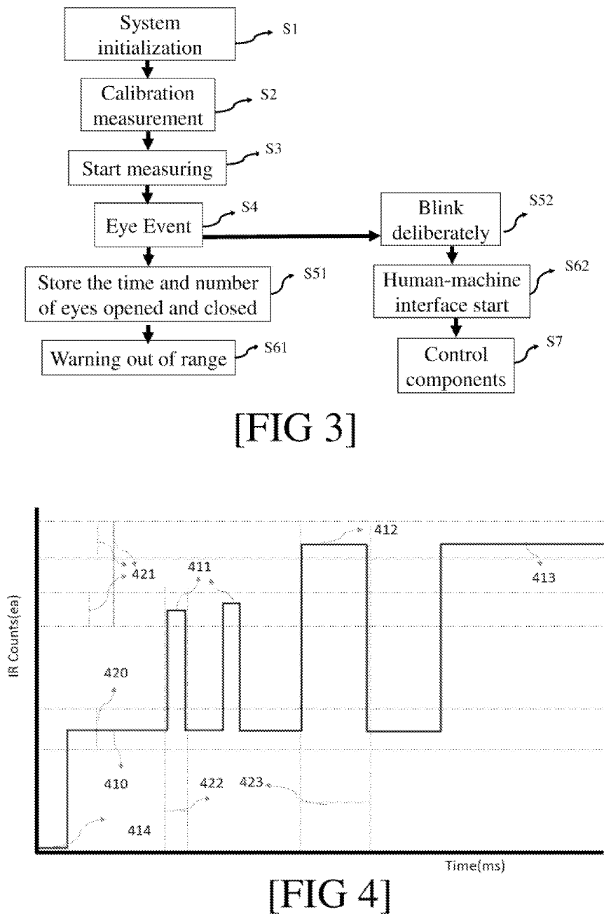

Refer to [FIG. 3] and [FIG. 4], when measurement program executed the system initializes step S1 (see the embedded system technical manual for details), set the infrared proximity sensor to detect. The setting of sensor's measurement frequency should higher than the unconscious blinking speed (100 ms) of the eye, preferably above 20 Hz. The calibration step S2 continuously collects and calculates the number of reflected infrared light pluses from eye by the receiver tube for an appropriate time (preferably 0.5 seconds). In normal conditions (the user with eye-opened 121), the number of reflected pulses would be value 410. It is the reference of the baseline and the eye-opened number range of pulses 420 when the eyes are opening. It shows another characteristic of the present invention: detecting eyelid opening according to the number of reflected light pulse counts, wherein the detection of eyelid opening is included determining the corresponding particular eye-opening state by the number of reflected pulse light data.

Referring to [FIG. 1] and [FIG. 4], eye closure can be subdivided into unconscious blinking closed eyes 122 and deliberate blinking closed eyes or long-term closed eyes 120. When the eyelids are closed due to unconscious blinking, the eyelids are not completely closed. So the measured distance is farther than the closed eye 412 of deliberate blinking and has a lower number of reflected pulses 411; the one of embodiment of the disclosure uses the deliberate eye-closing action 120 to collect the eye-closed number of infrared light reflected pulses 412, and then according to the experiment or the empirical value (eyelid thickness is about 0.5-2 mm) to get the value of the number of reflected infrared light pulses for unconscious closed eyes 122, which is a reference for the number of closed eye pulses range 421, and then shows another characteristic of the disclosure: detecting eyelid closing according to the number of reflected light pulse counts, wherein the detection of eyelid closing is included determining the corresponding particular eye closing state by the number of reflected infrared light pulses.

According to the determined particular eye opening and closing state, a particular eye state can be opened, unconscious closed, conscious closed or a sequence of specific eye opening and closing combinations. It can be used for computer calculation or signal transmission, such as an eye-opening action plus a conscious eye-closing action, and an eye-opening action is summed to a deliberate eye-blinking action. This blinking action can be used as control signals to form a human-machine interface.

Referring to [FIG. 4], the eye events of conscious blink, unconscious blink or long-term closed eyes are not only different in the distance change, but also in time duration; the duration of human unconscious blinking eyes closed and opened eyes is about 100 ms 422, so the closed eye duration 423 that exceeds the time 422 is related to a conscious blink event 412 or a long-term closed eye 413.

The time range for closing the eyes of a conscious blink event is preferably 100 ms to 2 s. If it exceeds the time range such as wave 413, means that the user is tired or entering into an abnormal activity such as a doze event.

The natural blink frequency of an adult is about 15 ea./min, so a blink cycle is about 4 seconds. If the eye opening that exceeds the normal blink cycle time is detected, it means that there may be evaporation of tears with insufficient replenishment and eye health risks. Eye events can be transmitted through computer calculations to transmit signals for warning step S61.

If the number of detected infrared light reflected pulses is around the zero counts 414 for a long time, it is defined as the user takes off device and enters a rest event.

The device can transmit a signal through computer calculations to record the usage time, and automatically switch to the power saving mode.

If the number of reflected infrared light pulses exceeds the sensing range of eyelid opening and closing, it is defined as a fixed position deviation event (HIVID minor shift or sliding), and the signal needs to be transmitted through computer calculations and then go back to the calibration step S2 again.

Disclosure can be expanded the scale of timing function, long period data can be collected and recorded by memory unit 502, in order to improve the understanding of the time change effect on eye care, such as daily, quarterly, or blink rate per person per year, and comparing with multi-users' eyes.

Previous shows another characteristic of the disclosure: calculating the time duration data based on the particular eye opening and closing state, and determine the corresponding particular eye event in the time duration data of the particular eye opening and closing state.

Referring to [FIG. 4], [FIG. 5], after the calibration step S2 done, enter the continuous measurement step S3, that is continue to collect the number of reflected infrared light pulses from the eye by the proximity sensor. If the system detects that the eye event S4 is an intentional blinking event, it transmits a signal through the computer operation to the corresponding step S52 and activates the human-machine interface S62: sends an preset on or off (0 and 1) signal through the processor program, the microcontroller and Bluetooth protocol 525 (see LED Button Service for details) can use this signal to allow users to control external components in a wireless or wired manner S7 and forms a basic human-machine interface S62.

In addition to detecting specific eye events, the system can continuously collect and store the frequency of eye opening and closing S51 for users to track eye usage.

The step S61 of warning out of range can use the vibration (motor 524), sound (speaker 523), light (LED522) alarms of the system to transmit signals through computer calculations to connect the system immediately to notify the user; It can also be wired or wirelessly to transmit data to a mobile phone or computer (see WIFI and Bluetooth UART Service for details) for real-time or long-term data analysis.

Previous shows another characteristic of the disclosure: at least one computing action to be performed based on the particular eye event and the particular eye opening and closing state.

Another embodiment of the disclosure is to use the light receiver 102 of the photoelectric proximity sensor 10 to measure the ambient light intensity (see the Ambient Light Sensor or Proximity sensor technical manual for details) data, no additional sensor is required. According to the study (The myopia boom, Nature. & Interventional Research on Myopia Prevention and Control), outdoor activities have a 30% benefit in controlling the degree of myopia. Students who spend more than one hour outdoors a day have a significant reduction in the proportion of myopia. Disclosure can combine with real-time detection of ambient light intensity and long-term statistics and record user activities and environmental conditions to track and control eye health. It shows characteristics of the disclosure: generating ambient light intensity data representing at least one type of eye environment by proximity sensor, and the type of eye environment is detected based on the ambient light intensity data. And determining the corresponding outdoor and indoor environments in the light intensity data; and the particular eye event is detected, which is further combined with the type of eye environment.

Disclosure can be combined with other sensors 512 for more applications, such as axis motion sensor 511 (see IMU Sensor Technical Manual for details), to detect the head movement. Head's axis data is the movement of mouse, eye's deliberate blinking event is button click. Wireless mouse function was provided for controlling external components 53 or systems by Bluetooth protocol 525 (see Bluetooth Human Interface Device Service).

Those who are familiar with the technology of the present disclosure should clearly understand that the present disclosure is not limited to the details of the above illustrative embodiments. The present disclosure can be implemented in other specific forms without departing from the basic attributes of the present disclosure. The embodiments are merely illustrative of the present disclosure. Instead of limiting the present disclosure, the present disclosure is based on the scope of the following clams, rather than based on the above description. The meaning of the scope of the following clams applied for and all variants in the equal scope belong to the scope of the present disclosure.

What is claimed is:

1. A detection and control method comprising:
at least one proximity sensor monitoring the change of the distance between the proximity sensor and at least one eye area of head, wherein the proximity sensor is used to generate the number of reflected light pulse counts representing the distance between the proximity sensor and the eye area; and
wherein the proximity sensor is firmly connected to the head; and
detecting eyelid opening and closing according to the number of reflected light pulse counts, wherein the detection of eyelid opening and closing is comprising determining the corresponding particular eye opening and closing state by the number of reflected light pulse counts; and
at least one computing action to be performed based on the particular eye opening and closing state.

2. The method of claim 1, at least one computing action to be performed further comprising:
at least one axis motion sensor monitoring the movement of the head, wherein the axis motion sensor is used to generate the data of axis change representing the movement of the head; and
wherein the particular eye opening and closing state which is further combined with the movement of the head.

3. The method of claim 1 is further comprising:
at least one wireless transmission function generating the wireless signal to at least one device; and
wherein the computing action is further combined with the wireless transmission function.

4. The method of claim 1, wherein the proximity sensor is further comprising:
generating ambient light intensity data representing at least one type of eye environment, and
the type of eye environment is detected based on the ambient light intensity data.

5. The method of claim 4, wherein the detection the type of eye environment is further comprising:
determining the corresponding outdoor and indoor environment in the ambient light intensity data; and
wherein the particular eye opening and closing state is detected, which is further combined with the type of eye environment.

6. The method of claim 1, wherein detecting eyelid opening and closing is further comprising:
determining at least one corresponding particular eye event within the number of reflected light pulse counts, wherein determining the particular eye event comprising:
calculating the time duration data based on the particular eye opening and closing state; and
determine the corresponding particular eye event in the time duration data of the particular eye opening and closing state; and
the computing action to be performed further comprising: at least one computing action to be performed based on the particular eye event.

7. The method of claim 6, wherein the proximity sensor is further comprising:
generating ambient light intensity data representing at least one type of eye environment, and
the type of eye environment is detected based on the ambient light intensity data, wherein the detection the type of eye environment is further comprising:
determining the corresponding outdoor and indoor environment in the ambient light intensity data; and
wherein the particular eye event is detected, which is further combined with the type of eye environment.

8. A detection and control system comprising:
at least one proximity sensor;
at least one fixing mechanism that firmly connects the proximity sensor with the head;
computer-readable media;
program instructions, which are stored in the computer-readable medium and can be processed by at least one processor to perform functions comprising:
monitoring the change of the distance between the proximity sensor and at least one eye area of head by the proximity sensor, wherein the proximity sensor is used to generate the number of reflected light pulse counts representing the distance between the proximity sensor and the eye area; and
detecting eyelid opening and closing according to the number of reflected light pulse counts, wherein the detection of eyelid opening and closing is comprising determining the corresponding particular eye opening and closing state by the number of reflected light pulse counts; and
causing at least one computing action to be performed based on the particular eye opening and closing state.

9. The method of claim 8 is further comprising:
at least one axis motion sensor monitoring the movement of the head, wherein the axis motion sensor is used to generate the data of axis change representing the movement of the head; and
wherein the particular eye opening and closing state which is further combined with the movement of the head.

10. The method of claim 8 is further comprising:
at least one wireless transmission function generating the wireless signal to at least one device; and
wherein the program instructions is further comprising: transferring signal wireless.

11. The method of claim 8, wherein the proximity sensor is further comprising:
generating ambient light intensity data representing at least one type of eye environment, and
the type of eye environment is detected based on the ambient light intensity data.

12. The method of claim 11, wherein the detection the type of eye environment is further comprising:
determining the corresponding outdoor and indoor environment in the light intensity data; and
wherein the particular eye opening and closing state is detected, which is further combined with the type of eye environment.

13. The method of claim 8, wherein detecting eyelid opening and closing is further comprising:
determining at least one corresponding particular eye event within the number of reflected light pulse counts, wherein determining the particular eye event comprising:
calculating the time duration data based on the particular eye opening and closing state; and
determine the corresponding particular eye event in the time duration data of the particular eye opening and closing state; and
the computing action to be performed is further comprising: at least one computing action to be performed based on the particular eye event.

14. The method of claim 13, wherein the proximity sensor is further comprising:
generating ambient light intensity data representing at least one type of eye environment, and
the type of eye environment is detected based on the ambient light intensity data, wherein the detection the type of eye environment is further comprising:
determining the corresponding outdoor and indoor environments in the light intensity data; and
wherein the particular eye event is detected, which is further combined with the type of eye environment.
